(19) United States
(12) Patent Application Publication
(10) Pub. No.: US 2014/0005556 A1
(43) Pub. Date: Jan. 2, 2014

(54) PHOTOACOUSTIC MEASURING APPARATUS

(71) Applicant: FUJIFILM Corporation, Tokyo (JP)

(72) Inventors: Kazuhiro HIROTA, Ashigarakami-gun (JP); Kazuhiro TSUJITA, Ashigarakami-gun (JP); Kaku IRISAWA, Ashigarakami-gun (JP)

(73) Assignee: FUJIFILM Corporation, Tokyo (JP)

(21) Appl. No.: 14/017,411

(22) Filed: Sep. 4, 2013

Related U.S. Application Data
(63) Continuation of application No. PCT/JP2012/001583, filed on Mar. 8, 2012.

(30) Foreign Application Priority Data
Mar. 10, 2011 (JP) 2011-052742
Feb. 13, 2012 (JP) 2012-028219

(51) Int. Cl. A61B 5/00; A61B 8/00

(52) U.S. Cl. CPC A61B 5/0095; A61B 8/4444
USPC 600/476

(57) ABSTRACT

A probe includes an acoustic wave transmitting section that transmits acoustic waves toward a subject, a light irradiating section that irradiates a light beam guided from a light source, and an acoustic wave detecting section that detects photoacoustic waves generated within the subject due to irradiation of the light beam onto the subject and reflected acoustic waves of the acoustic waves transmitted into the subject. In addition, a mode switching switch is provided on the probe. A control means switches operating modes between an operating mode in which the acoustic wave detecting section detects at least photoacoustic waves, and an operating mode in which the acoustic wave detecting section does not detect photoacoustic waves.

### TECHNICAL FIELD

The present invention is related to a photoacoustic measuring apparatus. More specifically, the present invention is related to a photoacoustic measuring apparatus that irradiates light onto a subject and detects acoustic waves generated within the subject by the irradiation of the light.

### BACKGROUND ART

The ultrasound examination method is known as an image examination method that enables examination of the state of the interior of living organisms in a non invasive manner. Ultrasound examination employs an ultrasound probe capable of transmitting and receiving ultrasonic waves. When the ultrasonic waves are transmitted to a subject (living organism) from the ultrasound probe, the ultrasonic waves propagate through the interior of the living organisms, and are reflected at interfaces among tissue systems. The ultrasound probe receives the reflected ultrasonic waves and images the state of the interior of the subject, by calculating distances based on the amounts of time that the reflected ultrasonic waves return to the ultrasound probe.

Photoacoustic imaging, which images the interiors of living organisms utilizing the photoacoustic effect, is also known. Generally, in photoacoustic imaging, pulsed laser beams are irradiated into living organisms. Biological tissue within the living organisms that absorbs the energy of the pulsed laser beams generates ultrasonic waves (photoacoustic signals) by volume expansion thereof due to heat. An ultrasound probe or the like detects the photoacoustic signals, and constructs photoacoustic images based on the detected signals, to enable to enable visualization of the living organisms based on the photoacoustic signals.

An apparatus capable of generating both photoacoustic images and ultrasound images is disclosed in Japanese Unexamined Patent Publication No. 2005-218684, for example. In Japanese Unexamined Patent Publication No. 2005-218684, a keyboard provided on an operating panel, a track ball, a mouse, and the like are employed to input a command to initiate collection of photoacoustic image data.

### DISCLOSURE OF THE INVENTION

In an apparatus capable of generating photoacoustic images and ultrasound images, it is possible to display (generate) three types of images. The three types of images are: ultrasound images; photoacoustic images; and images in which ultrasound images and photoacoustic images are overlapped on each other. In Japanese Unexamined Patent Publication No. 2005-218684, the types of images are switched by operating the keyboard provided on the operating panel, the track ball, the mouse, and the like. However, in such a case, an operator must change the direction that they are facing and to let go of a probe that they are holding in their hand, in order to operate the operating panel, which is troublesome.

The present invention has been developed in view of the foregoing circumstances. It is an object of the present invention to provide a photoacoustic measuring apparatus in which switching of displayed images by an operator is facilitated.

In order to achieve the above object, the present invention provides a photoacoustic measuring apparatus, comprising:

a light source;

an acoustic wave transmitting section that transmits acoustic waves toward a subject;

a light irradiating section that irradiates a light beam guided from the light source toward the subject;

a probe including an acoustic wave detecting section that detects photoacoustic waves generated within the subject due to irradiation of the light beam onto the subject and detects reflected acoustic waves of the acoustic waves transmitted into the subject;

a mode switching switch provided on the probe; and

a control section that switches among operating modes in which the acoustic wave detecting section detects at least the photoacoustic waves, and operating modes in which the acoustic wave detecting section does not detect the photoacoustic waves, in response to operation of the mode switching switch.

In the present invention, a configuration may be adopted, wherein:

the operating modes include a first operating mode in which the acoustic wave detecting section detects the photoacoustic waves, a second operating mode in which the acoustic wave detecting section detects the reflected acoustic waves, and a third operating mode in which the acoustic wave detecting section detects both the photoacoustic waves and the reflected acoustic waves; and

the control section switches among the first through third operating modes each time that the mode switching switch is operated.

It is preferable for the control section to set the operating mode to that in which the acoustic wave detecting section does not detect the photoacoustic waves in an initial state.

An alternate action push button switch may be employed as the mode switching switch.

The photoacoustic measuring apparatus may further comprise:

an image generating section that generates photoacoustic images and acoustic images based on detected signals of the photoacoustic waves and detected signals of the reflected acoustic waves.

The probe may include at least one of the acoustic wave transmitting section and the light irradiating section.

A configuration may be adopted, wherein:

the light source is capable of outputting light beams having a plurality of different wavelengths;

the photoacoustic measuring apparatus further comprises a wavelength selecting switch for selecting the wavelength of a light beam to be irradiated onto a subject; and

the control section controls the wavelength of the light beam to be output from the light source in response to operation of the wavelength selecting switch, in addition to switching of the operating modes.

The wavelength selecting switch may be provided on the probe.

A configuration may be adopted, wherein:

the control section causes the light source to output a light beam having a first wavelength, to output a light beam having a second wavelength different from the first wavelength, or to alternately output the light beam having the first wavelength and the light beam having the second wavelength, in response to operation of the wavelength selecting switch.

A slide switch may be employed as the wavelength selecting switch.

The photoacoustic measuring apparatus of the present invention may further comprise:

a contact state judging section that judges whether the probe is in contact with a subject. In this case, a light beam may be irradiated onto the subject when the contact state judging section judges that the probe is in contact with the subject.

The contact state judging section may judge whether the probe is in contact with the subject, based on detected signals of the reflected acoustic waves.

The present invention also provides a photoacoustic measuring apparatus, comprising:

a light source;

an acoustic wave transmitting section that transmits acoustic waves toward a subject;

a light irradiating section that irradiates a light beam guided from the light source toward the subject;

a probe including an acoustic wave detecting section that detects photoacoustic waves generated within the subject due to irradiation of the light beam onto the subject and detects reflected acoustic waves of the acoustic waves transmitted into the subject;

a mode switching switch for switching operating modes;

a wavelength selecting switch for selecting the wavelength of the light beam to be output from the light source; and

a control section that switches among operating modes in which the acoustic wave detecting section detects at least the photoacoustic waves, and operating modes in which the acoustic wave detecting section does not detect the photoacoustic waves in response to operation of the mode switching switch, and controls the wavelength of the light beam which is output from the light source in response to operation of the wavelength selecting switch;

at least one of the mode switching switch and the wavelength selecting switch being provided on the probe.

In the photoacoustic measuring apparatus of the present invention, both the mode switching switch and the wavelength selecting switch may be provided on the probe.

An alternate action push button switch may be employed as the mode switching switch.

An alternate configuration may be adopted, wherein:

only the wavelength selecting switch is provided on the probe; and

the mode switching switch is a footswitch to be operated by the foot of an operator. An alternate action switch may be employed as the footswitch.

A slide switch may be employed as the wavelength selecting switch.

The photoacoustic measuring apparatus of the present invention is provided with a mode switching switch on the probe. Operating modes are switched between an operating mode that includes detection of photoacoustic signals and an operating mode that does not include detection of photoacoustic signals by operating the switch. An operator can switch images to be generated by operating the switch provided on the probe, and displayed images can be easily switched.

### BRIEF DESCRIPTION OF THE DRAWINGS

FIG. 1 is a block diagram that illustrates a photoacoustic measuring apparatus according to a first embodiment of the present invention.

FIG. 2 is a diagram that illustrates the outer appearance of a probe.

FIG. 3 is a flow chart that illustrates the steps of an operational procedure when an operating mode is an ultrasound image generating mode.

FIG. 4 is a flow chart that illustrates the steps of an operational procedure when an operating mode is a photoacoustic image generating mode.

FIG. 5 is a flow chart that illustrates the steps of an operational procedure when an operating mode is that which generates both types of images.

FIG. 6 is a diagram that illustrates an example of an ultrasound image.

FIG. 7 is a diagram that illustrates an example of a photoacoustic image.

FIG. 8 is a diagram that illustrates an example of an image in which a photoacoustic image and an ultrasound image are overlapped.

FIG. 9 is a block diagram that illustrates a photoacoustic measuring apparatus according to a second embodiment of the present invention.

FIG. 10 is a diagram that illustrates the outer appearance of a probe.

FIG. 11 is a block diagram that illustrates the configuration of a laser unit.

FIG. 12 is a diagram that illustrates an example of the configurations of a wavelength selecting element, a drive means, and a driving state detecting means.

FIG. 13 is a block diagram that illustrates a photoacoustic measuring apparatus according to a third embodiment of the present invention.

FIG. 14 is a block diagram that illustrates a photoacoustic measuring apparatus according to a modification to the present invention.

FIG. 15 is a diagram that illustrates the outer appearance of a probe of the modification.

### BEST MODE FOR CARRYING OUT THE INVENTION

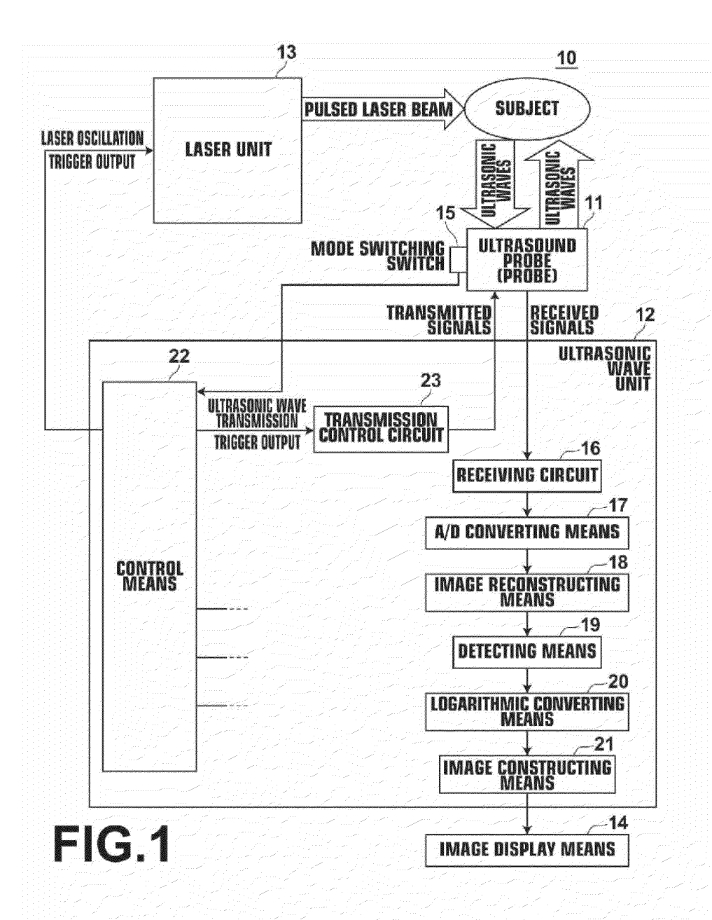

Hereinafter, embodiments of the present invention will be described in detail with reference to the attached drawings. FIG. 1 illustrates a photoacoustic measuring apparatus 10 according to a first embodiment of the present invention. The photoacoustic measuring apparatus 10 includes: an ultrasound probe (probe) 11; an ultrasonic wave unit 12; and a light source (laser unit) 13. The laser unit 13 is a light source, and generates a laser beam to be irradiated onto subject. The wavelength of the laser beam may be set as appropriate according to targets of observation. The laser beam output by the laser unit 13 is guided to the probe 11 by a light guiding means such as an optical fiber.

The probe 11 includes a light irradiating section that irradiates the laser beam guided thereto from the laser unit 13 onto subjects. In addition, the probe 11 includes an acoustic wave transmitting section that outputs (transmits) acoustic waves (typically, ultrasonic waves) to subjects and an acoustic wave detecting section that detects (receives) acoustic waves reflected by the subjects. The probe 11 has a plurality of ultrasonic transducers which are arranged one dimensionally, for example. The probe outputs ultrasonic waves from the plurality of ultrasonic transducers when generating ultrasound images and detects reflected acoustic waves (hereinafter, also referred to as “reflected acoustic signals”), for example. The probe 11 detects acoustic waves (hereinafter, also referred to as “photoacoustic signals”) which are generated by targets of measurement within subjects absorbing the laser beam output by the laser unit 13 when generating photoacoustic images. Note that it is only necessary for the probe 11 to include the acoustic wave detecting section at least, and one or both of the acoustic wave transmitting section and the light irradiating section ay be provided outside the probe 11.

The probe 11 has a mode switching switch 15 for switching the operating mode of the apparatus. An alternate action push button switch may be employed as the mode switching switch 15, for example. The mode switching switch 15 is utilized to switch between operating modes in which the acoustic wave detecting section of the probe 11 detects at photoacoustic waves at least, and operating modes in which the acoustic wave detecting section does not detect photoacoustic waves. The operating modes include a first operating mode in which the acoustic wave detecting section detects photoacoustic waves, a second operating mode in which the acoustic wave detecting section detects reflected acoustic waves, and a third operating mode in which the acoustic wave detecting section detects both photoacoustic waves and reflected acoustic waves. An operator, such as a physician, can switch among detection of photoacoustic signals, detection of reflected acoustic signals, and detection of both photoacoustic signals and reflected acoustic signals, by pressing the mode switching switch 15.

The ultrasonic wave unit 12 has a detected signal processing section that processes detected signals of photoacoustic waves and reflected acoustic waves detected by the probe. The signal processing section is constituted as an image generating section that generates tomographic images based on detected signals of acoustic waves detected by the probe 11. In FIG. 1, an image reconstructing means 18, a detecting means 19, a logarithmic converting means 20, and an image constructing means 21 constitute the image generating section. The functions of each component of the image generating section may be realized by a computer executing processes according to predetermined programs. The image generating section generates first tomographic images (photoacoustic images) based on photoacoustic signals received by the probe 11 as well as second tomographic images (ultrasound images) based on reflected acoustic signals received by the probe 11.

The image reconstructing means 18 generates data corresponding to each line of tomographic images, based on detected signals of acoustic waves detected by the plurality of ultrasonic wave transducers of the probe 11. The image reconstructing means 18 adds data from 64 ultrasonic transducers of the probe 11 at delay times corresponding to the positions of the ultrasonic transducers, to generate data corresponding to a single line (delayed addition method), for example. Alternatively, the image reconstructing means 18 may execute image reconstruction by the CBP (Circular Back Projection) method. As further alternatives, the image reconstructing means 18 may execute image reconstruction by the Hough transform method or Fourier transform method.

The detecting means 19 outputs envelope curves for data corresponding to each line output by the image reconstructing means 18. The logarithmic converting means 20 logarithmically converts the envelope curves output by the detecting means 19, to widen the dynamic ranges thereof. The image constructing means 21 generates tomographic images by converting the positions of acoustic waves (peak portions) along a temporal axis to positions in the depth direction of the tomographic images. An image display means 14 displays the tomographic images generated by the image constructing means 21 on a display monitor or the like.

A control means 22 (control section) controls each component within the ultrasonic wave unit 12. The control exerted by the control means 22 includes switching of operating modes. The control means 22 switches among operating modes in which the acoustic wave detecting section of the probe 11 detects photoacoustic waves at least and operating modes in which the acoustic wave detecting section does not detect photoacoustic waves, in response to operation of the mode switching switch 15. The control means 22 switches among an operating mode that detects photoacoustic waves and generates photoacoustic images, an operating mode that detects reflected acoustic waves and generates ultrasound images, and an operating mode that detects both photoacoustic waves and reflected acoustic waves and generates both photoacoustic images and ultrasound images, for example.

The control means 22 sets the operating mode to that which does not detect photoacoustic waves, specifically, an operating mode that generates ultrasound images, in an initial state, that is, a state in which the mode switching switch 15 has not been depressed once, for example. The control means 22 switches operating modes every time that the mode switching switch 15 is operated, for example. If the mode switching switch 15 is depressed while the operating mode is the ultrasound image generating mode, the control means 22 switches the operating mode to a photoacoustic image generating mode. If the mode switching switch 15 is depressed again, the control means 22 switches the operating mode from the photoacoustic image generating mode to a mode that generates both ultrasound images and photoacoustic images. If the mode switching switch 15 is depressed while in the mode that generates both types of images, the operating mode is returned to the ultrasound image generating mode. Thereafter, the control means 22 sequentially switches the operating mode in order from the ultrasound image generating mode, the photoacoustic image generating mode, and the mode that generates both ultrasound images and photoacoustic images.

In addition, the control means 22 outputs an ultrasonic wave transmission trigger to a transmission control circuit 23 when transmitting ultrasonic waves to subjects. When the trigger signal is received, the transmission control circuit 23 causes the probe 11 to transmit ultrasonic waves. The control means 22 controls the sampling initiation timing of an A/D converting means 17 synchronized with the ultrasonic wave transmission trigger signal. Meanwhile, the control means 22 transmits a laser oscillation trigger signal to the laser unit 13 in the case that the operating mode is a mode that generates photoacoustic images or a mode that generates ultrasound images and photoacoustic images. When the trigger signal is received, the laser unit 13 performs laser oscillation and outputs a laser beam. The control means 22 controls the sampling initiation timing of an A/D converting means 17 synchronized with the laser oscillation trigger signal.

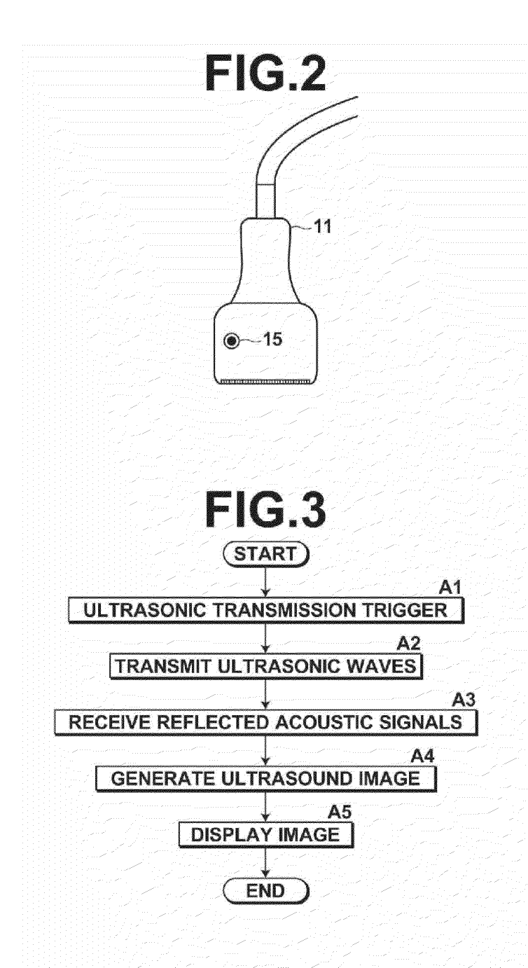

FIG. 2 illustrates the outer appearance of the probe 11. An operator holds the probe 11 in their hand and causes the surface thereof at which the ultrasonic transducers are arranged to contact a portion to be observed. The mode switching switch 15 is provided at a portion of the probe 11 at which the thumb is positioned when the probe 11 is held in the operator's hand. The operator sets a desired operating mode by operating the mode switching switch 15 as appropriate.

FIG. 3 illustrates the steps of an operational procedure when the operating mode is a mode that generates ultrasound images. The control means 22 outputs an ultrasonic wave transmission trigger signal to the transmission control circuit 23 (step A1). The probe 11 transmits ultrasonic waves into the body of a subject (step A2). The probe 11 receives reflected acoustic signals which are reflected within the body of the subject (Step A3). The image generating means 23 within the ultrasonic wave unit 12 generates an ultrasound image based on the reflected acoustic signals (step A4). The image display means 14 displays the ultrasound image generated by the ultrasonic wave unit 12 on a display screen (step A5).

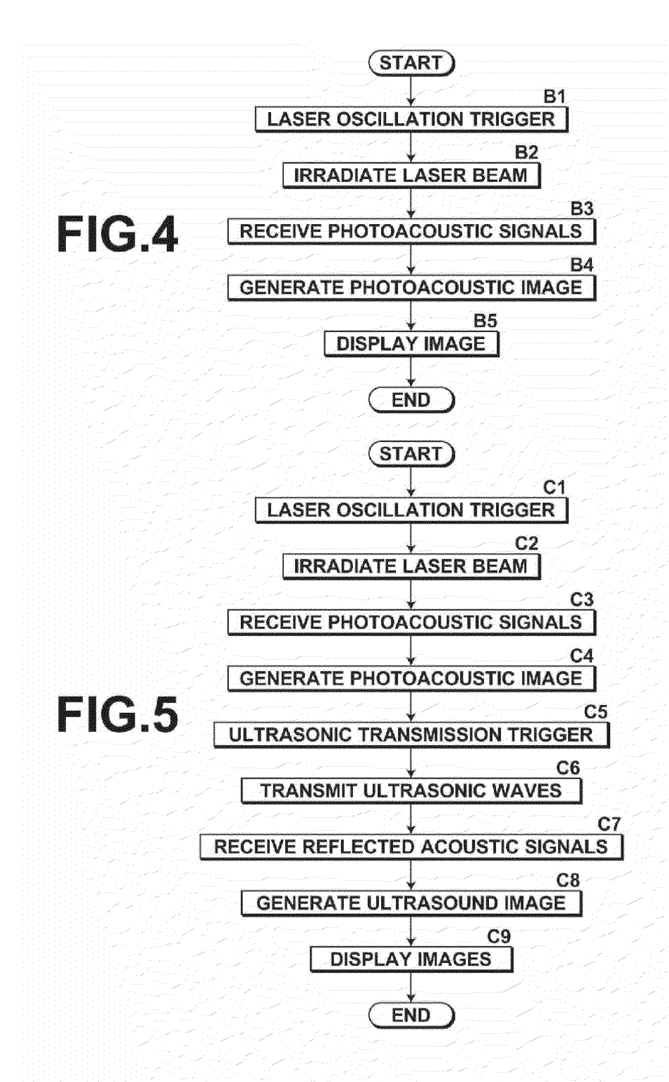

FIG. 4 illustrates the steps of an operational procedure when the operating mode is a mode that generates photoacoustic images. The control means 22 outputs a laser oscillation trigger signal to the laser unit 13 (step B1). The laser unit 13 outputs a pulsed laser beam, and the pulsed laser beam output by the laser unit 13 is irradiated onto a subject from the probe 11 (step B2). The probe 11 receives photoacoustic signals which are generated within the body of the subject due to irradiation of the laser beam (Step B3). The image generating means 23 within the ultrasonic wave unit 12 generates an ultrasound image based on the photoacoustic signals (step B4). The image display means 14 displays the photoacoustic image generated by the ultrasonic wave unit 12 on a display screen (step B5).

FIG. 5 illustrates the steps of an operational procedure when the operating mode is a mode that generates photoacoustic images and ultrasound images. The control means 22 outputs a laser oscillation trigger signal to the laser unit 13 (step C1). The laser unit 13 outputs a pulsed laser beam, and the pulsed laser beam output by the laser unit 13 is irradiated onto a subject from the probe 11 (step C2). The probe 11 receives photoacoustic signals which are generated within the body of the subject due to irradiation of the laser beam (Step C3). The image generating means 23 within the ultrasonic wave unit 12 generates an ultrasound image based on the photoacoustic signals (step C4).

Thereafter, the control means 22 outputs an ultrasonic wave transmission trigger signal to the transmission control circuit 23 (step C5). The probe 11 transmits ultrasonic waves into the body of the subject (step C6). The probe 11 receives reflected acoustic signals which are reflected within the body of the subject (Step C7). The image generating means 23 within the ultrasonic wave unit 12 generates an ultrasound image based on the reflected acoustic signals (step C8). The image display means 14 displays the photoacoustic image and the ultrasound image generated by the ultrasonic wave unit 12 on a display screen (step C9). The image display means 14 displays the photoacoustic image and the ultrasound image in an overlapping manner, for example. Note that in FIG. 5, the photoacoustic image was generated first, and then the ultrasound image was generated next. Alternatively, the ultrasound image may be generated first, and then the photoacoustic image may be generated thereafter.

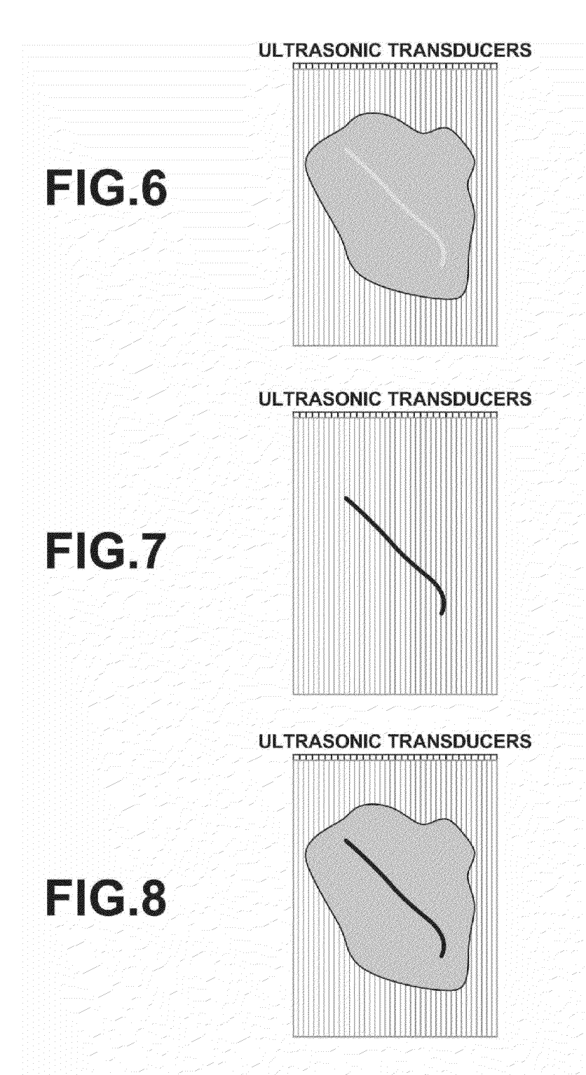

FIG. 6 is a diagram that illustrates an ultrasound image, FIG. 7 is a diagram that illustrates a photoacoustic image, and FIG. 8 is a diagram that illustrates an example of an image in which a photoacoustic image and an ultrasound image are overlapped. In each of these figures, the horizontal direction corresponds to the direction in which the ultrasonic transducers are arranged, and the vertical direction corresponds to a depth direction. When the operating mode is that which generates ultrasound images, the image display means 14 displays an ultrasound image such as that illustrated in FIG. 6. When the operating mode is that which generates photoacoustic images, the image display means 14 displays a photoacoustic image such as that illustrated in FIG. 7. When the operating mode is that which generates photoacoustic images and ultrasound images, the image display means 14 displays an image in which a photoacoustic image and an ultrasound image are overlapped, such as that illustrated in FIG. 8.

In the present embodiment, the control means switches the operating mode of the photoacoustic measuring apparatus 10 each time that the mode switching switch 15 is operated. For example, if the mode switching switch 15 is operated once while the photoacoustic measuring apparatus 10 is operating in the ultrasound image generating mode, the operating mode is switched to the photoacoustic image generating mode, and observation of photoacoustic images becomes possible. In the present embodiment, the mode switching switch 15 for switching operating modes is provided on the probe. Therefore, operators can switch images which are observed without operating an operating panel or the like, and switching of images which are displayed is facilitated. It is preferable for mode of the photoacoustic measuring apparatus 10 to be that which does not include generation of photoacoustic images in an initial state. If this configuration is adopted, sudden irradiation of a laser beam can be prevented.

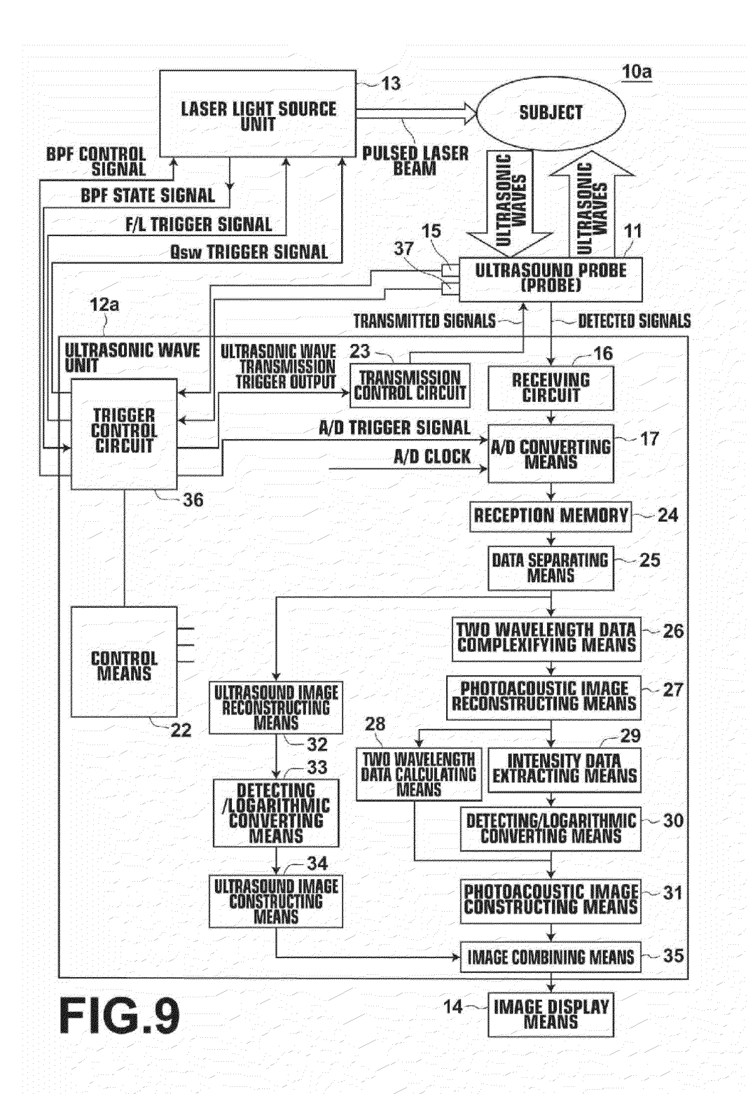

Next, a second embodiment of the present invention will be described. FIG. 9 illustrates a photoacoustic measuring apparatus 10a according to the second embodiment of the present invention. The photoacoustic measuring apparatus 10a is equipped with an ultrasound probe (probe) 11, an ultrasonic wave unit 12a, and a light source (laser unit) 13. The ultrasonic wave unit 12a has a receiving circuit 16, an A/D converting means 17, a photoacoustic image generating means 27, a photoacoustic image constructing means 31, a control means 22, a transmission control circuit 23, a reception memory 24, a data separating means 25, a two wavelength data complexifying means 26, a two wavelength data calculating means 28, an intensity data extracting means 29, a detecting/logarithmic converting means 30, an ultrasound image reconstructing means 32, a detecting/logarithmic converting means 33, an ultrasound image constructing means 34, an image combining means 35, and a trigger control circuit 36.

In the present embodiment, the laser unit 13 is configured to be capable of outputting a plurality of laser beams having different wavelengths from each other. In the case that light beams having a plurality of wavelengths are irradiated onto subjects, wavelength dependent properties of light absorption characteristics for light absorbers within subjects are utilized to generate photoacoustic images in which arteries and veins can be distinguished, for example. The switching of operating modes of the apparatus using a mode switching switch 15 is the same as in the first embodiment. That is, the operating mode of the apparatus is switched from an operating mode that generates only ultrasound images, an operating mode that generates only photoacoustic images, and an operating mode that generates both types of images, each time that the mode switching switch constituted by an alternate action push button switch is depressed.

Pulsed laser beams output from the laser unit 13 are guided to the probe 11 by a light guiding means such as an optical fiber, and then are irradiated onto a subject from the probe 11. The following description will mainly be of a case in which the laser unit is capable of outputting a pulsed laser beam having a first wavelength and a pulsed laser beam having a second wavelength.

A case will be considered in which the first wavelength (central wavelength) is approximately 750 nm, and the second wavelength is approximately 800 nm. The molecular absorption coefficient of oxidized hemoglobin (hemoglobin bound to oxygen: oxy-Hb), which is contained in human arteries, for a wavelength of 750 nm is greater than that for a wavelength of 800 nm. Meanwhile, molecular absorption coefficient of deoxidized hemoglobin (hemoglobin not bound to oxygen: deoxy-Hb), which is contained in veins, for a wavelength of 750 nm is less than that for a wavelength of 800 nm. Photoacoustic signals from arteries and photoacoustic signals from veins can be distinguished by checking the relative intensities of photoacoustic signals obtained for a wavelength of 800 nm and photoacoustic signals obtained for a wavelength of 750 nm, utilizing these characteristics.

The probe 11 has a light irradiating section that irradiates light onto subjects, an acoustic wave transmitting section that transmits acoustic waves toward subjects, and an acoustic wave detecting section that detects acoustic waves (photoacoustic waves and reflected acoustic waves) from subjects. The receiving circuit 16 receives detected signals of acoustic waves received by the probe 11. The A/D converting means 17 samples the detected signals received by the receiving circuit 16. The A/D converting means 17 samples the ultrasonic signals at a predetermined sampling period synchronized with an A/D clock signal, for example. The A/D converting means 17 stores reflected acoustic data obtained by sampling reflected acoustic signals and photoacoustic data obtained by sampling photoacoustic signals in the reception memory 24.

The control means 22 and the trigger control circuit 36 constitute a control section. The control means 22 controls each of the components within the ultrasonic wave unit 12a. The trigger control circuit 36 outputs a light trigger signal to the laser unit 13 when the operating mode of the apparatus is an operating mode that includes photoacoustic image generation. The light trigger signal corresponds to the laser oscillation trigger signal of the first embodiment. The trigger control circuit 36 first outputs a flash lamp trigger signal, then outputs a Q switch trigger signal thereafter. The laser unit 13 pumps a laser medium in response to the flash lamp trigger signal, and outputs a pulsed laser beam in response to the Q switch trigger signal. Note that the trigger control circuit 36 may be a portion of the control means 22.

The timing for the Q switch trigger may be generated within the laser unit 13 instead of the Q switch trigger being transmitted to the laser unit 13 from the trigger control circuit 36. In this case, a signal that indicates that a Q switch has been turned ON may be transmitted to the ultrasonic wave unit 12a from the laser unit 13. Here, the light trigger signal is a concept that includes at least one of the flash lamp trigger signal and the Q switch trigger signal. The Q switch trigger signal corresponds to the light trigger signal in the case that the trigger control circuit 36 outputs the Q switch trigger signal. The flash lamp trigger signal corresponds to the light trigger signal in the case that the laser unit 13 generates the timing of the Q switch trigger.

When the operating mode of the apparatus is an operating mode that includes ultrasound image generation, the trigger control circuit 36 outputs an ultrasonic wave trigger signal that commands acoustic wave transmission to the transmission control circuit 23. When the trigger signal is received, the transmission control circuit 23 causes the probe 11 to transmit acoustic waves (ultrasonic waves). When the operating mode of the apparatus is an operating mode that includes both photoacoustic image generation and ultrasound image generation, the trigger control circuit 36 outputs a light trigger signal and an ultrasonic wave trigger signal in a predetermined order. For example, the trigger control circuit 36 outputs a light trigger signal first, and then outputs an ultrasonic wave trigger signal. Irradiation of a laser beam and detection of photoacoustic signals are performed by the light trigger signal being output, and transmission of ultrasonic waves toward a subject and detection of reflected acoustic signals are performed thereafter by output of the ultrasonic wave trigger signal. The trigger control circuit 36 outputs a sampling trigger signal that commands initiation of sampling to the A/D converting means 17 after outputting the light trigger signal or the ultrasonic wave trigger signal. When the sampling trigger signal is received, the A/D converting means 17 initiates sampling of photoacoustic signals or reflected acoustic signals. The A/D converting means 17 stores the sampled photoacoustic signals and the sampled reflected acoustic signals in the reception memory 24.

Note that in the case that both a photoacoustic image and an ultrasound image are generated, photoacoustic signals and reflected acoustic signals may be continuously sampled instead of being sampled individually. For example, the trigger control circuit 36 outputs the ultrasonic wave trigger signal at a timing at which detection of photoacoustic signals is completed following output of the light trigger signal. At this time, the A/D converting means 17 continuously executes sampling without interrupting sampling of detection signals of photoacoustic waves. In other words, the trigger control circuit 36 outputs the ultrasonic wave trigger signal in a state in which the A/D converting means 17 is continuously sampling detected signals of acoustic waves. The acoustic waves detected by the probe 11 change from photoacoustic waves to reflected acoustic waves, by the probe transmitting ultrasonic waves in response to the ultrasonic wave trigger signal. The A/D converting means 17 continuously samples the photoacoustic waves and the reflected acoustic waves, by continuing sampling detected signals of detected acoustic waves. Both the sampled photoacoustic signals and the sampled reflected acoustic signals may be stored in a common reception memory 24.

If photoacoustic waves and reflected acoustic waves are generated at the same position in the depth direction of a subject, time is necessary for acoustic waves transmitted from the probe 11 to propagate to this position in the case of reflected acoustic waves. Therefore, the amount of time from acoustic wave transmission to reflected acoustic wave detection will be double the amount of time from light irradiation to photoacoustic wave detection. When generating ultrasound images, a ½ resampling means that resamples reflected acoustic signals into ½ may be provided, and ultrasound images in which reflected acoustic signals are compressed in ½ on a temporal axis may be generated. Alternatively, the sampling rate may be decreased to half, for example, two 20 MHz from 40 MHz, at a timing when detected signals of reflected acoustic waves are sampled.

Sampling of photoacoustic signals is repeated for the number of wavelengths of light output by the laser unit 13. For example, first, the light beam having the first wavelength is irradiated onto a subject from the laser unit 13, and first photoacoustic signals (first photoacoustic data) detected by the probe 11 when the pulsed laser beam having the first wavelength is irradiated onto the subject are stored in the reception memory 24. Next, the light beam having the second wavelength is irradiated onto the subject from the laser unit 13, and second photoacoustic signals (second photoacoustic data) detected by the probe 11 when the pulsed laser beam having the second wavelength is irradiated onto the subject are stored in the reception memory 24. In the case that photoacoustic signals and reflected acoustic signals are continuously sampled, sampling of the photoacoustic signals and the reflected acoustic signals may be repeated for the number of wavelengths. For example, reflected acoustic data may be stored in the reception memory 24 continuous with the first photoacoustic data, and reflected acoustic data may be stored in the reception memory 24 continuous with the then photoacoustic data.

The data separating means 25 separates the ultrasound data, the first photoacoustic data, and the second photoacoustic data, which are stored in the reception memory 24. The data separating means 25 provides the first and second photoacoustic data to the two wavelength data complexifying means 26. The two wavelength data complexifying means 26 generates complex number data, in which one of the first photoacoustic signals and the second photoacoustic signals is designated as a real part, and the other is designated as an imaginary part. Hereinafter, a case will be described in which the two wavelength data complexifying means 26 designates the first photoacoustic signals as the real part and the second photoacoustic signals as the imaginary part.

The complex number data, which are the photoacoustic data, are input to a photoacoustic image reconstructing means 27 from the two wavelength data complexifying means 26. The photoacoustic image reconstructing means 27 reconstructs the photoacoustic data. The photoacoustic image reconstructing means 27 reconstructs images from the input complex number data by the Fourier transform method (FTA method). Known techniques, such as that disclosed in J. I. Sperl et al., “Photoacoustic image reconstruction: a quantitative analysis”, SPIE-OSA, Vol. 6631, 663103, may be applied to image reconstruction by the Fourier transform method. The photoacoustic image reconstructing means 27 inputs data, which have undergone Fourier transform and represent reconstructed images, to the intensity data extracting means 29 and the wavelength data calculating means 28.

The two wavelength data calculating means 28 extracts the relative signal intensities between the photoacoustic data corresponding to each wavelength. In the present embodiment, the reconstructed images reconstructed by the photoacoustic image reconstructing means 27 are input to the two wavelength data calculating means 28. The two wavelength data calculating means 28 extracts phase data that represent which of the real part and the imaginary part is larger and by how much, by comparing the real part and the imaginary part of the input data, which are complex number data. When the complex number data is represented by X+iY, for example, the two wavelength data calculating means 28 generates θ=tan⁻¹(Y/X) as the phase data. Note that θ=90° in the case that X−0. When the first photoacoustic data (X) that constitutes the real part and the second photoacoustic data (Y) that constitutes the imaginary part are equal, the phase data is θ=45°. The phase data becomes closer to θ=0° as the first photoacoustic data is relatively larger, and becomes closer to θ=90° as the second photoacoustic data is relatively larger.

The intensity data extracting means 29 generates intensity data that represent signal intensities, based on the photoacoustic data corresponding to each wavelength. In the present embodiment, the reconstructed images reconstructed by the photoacoustic image reconstructing means 27 are input to the intensity data extracting means 29. The intensity data extracting means 29 generates the intensity data from the input data, which are complex number data. When the complex number data is represented by X+iY, for example, the intensity data extracting means 29 extracts (X²+Y²)^{1/2} as the intensity data. The detecting/logarithmic converting means 30 generates envelope curves of data that represent intensity data extracted by the intensity data extracting means 29, and logarithmically converts the envelope curves to widen the dynamic ranges thereof.

The phase data from the two wavelength data calculating means 28 and the intensity data, which have undergone the detection/logarithmic conversion process administered by the detecting/logarithmic converting means 30, are input to the photoacoustic image constructing means 31. The photoacoustic image constructing means 31 generates a photoacoustic image, which is a distribution image of light absorbers, based on the input phase data and intensity data. The photoacoustic image constructing means 31 determines the brightness (gradation value) of each pixel within the distribution image of light absorbers, based on the input intensity data, for example. In addition, the photoacoustic image constructing means 31 determines the color (display color) of each pixel within the distribution image of light absorbers, based on the phase data, for example. The photoacoustic image constructing means 31 employs a color map, in which predetermined colors correspond to a phase range from 0° to 90°, to determine the color of each pixel based on the input phase data for example for example.

Here, the phase range from 0° to 45° is a range in which the first photoacoustic data is greater than the second photoacoustic data. Therefore, the source of the photoacoustic signals may be considered to be arteries, through which blood that mainly contains oxidized hemoglobin having greater absorption with respect to a wavelength of 756 nm than a wavelength of 798 nm flows. Meanwhile, the phase range from 45° to 90° is a range in which the second photoacoustic data is greater than the first photoacoustic data. Therefore, the source of the photoacoustic signals may be considered to be veins, through which blood that mainly contains deoxidized hemoglobin having lower absorption with respect to a wavelength of 798 nm than a wavelength of 756 nm flows.

Therefore, a color map, in which a phase of 0° corresponds to red that gradually becomes colorless (white) as the phase approaches 45°, and a phase of 90° corresponds to blue that gradually becomes white as the phase approaches 45°, is employed. In this case, portions corresponding to arteries within the photoacoustic image can be displayed red, and portions corresponding to veins can be displayed blue. A configuration may be adopted, wherein the intensity data are not employed, the gradation values are set to be constant, and portions corresponding to arteries and portions corresponding to veins are merely separated by colors according to the phase data.

Note that in the case that a light beam having a single wavelength is irradiated onto the subject, the complexifying process by the two wavelength complexifying means and extraction of phase data by the two wavelength data calculating means 28 are unnecessary. In the case that a light beam having a single wavelength is irradiated onto the subject, a photoacoustic image may be generated based on intensity data extracted by the intensity data extracting means 29.

Meanwhile, the data separating means 25 provides the separated reflected acoustic data to the ultrasound image reconstructing means 32. The ultrasound image reconstructing means 32 generates data corresponding to each line of an ultrasound image, which is a tomographic image, based on the reflected acoustic signals (reflected acoustic data). The detecting/logarithmic converting means 33 generates envelope curves of data corresponding to each line output by the ultrasound image reconstructing means 32, and logarithmically converts the envelope curves to widen the dynamic ranges thereof. The ultrasound image constructing means 34 generates an ultrasound image based on the data corresponding to each line, on which logarithmic conversion has been administered.

The image combining means 35 combines the photoacoustic images generated by the photoacoustic image constructing means 31 and the ultrasound image generated by an ultrasound image constructing means 34. The combined image is displayed by an image display means 14. It is also possible for the image display means 14 to display the photoacoustic images and the ultrasound image arranged next to each other without combining the images, or to switch between display of the photoacoustic image and the ultrasound image.

The wavelength selecting switch 37 is a switch for selecting which wavelength of light, from among the plurality of wavelengths capable of being output by the laser unit 13, is to be output. For example, a user may select from among the first wavelength, the second wavelength, and alternate output of the first wavelength and the second wavelength when generating photoacoustic images, by operating the wavelength selecting switch 37. The trigger control circuit 36 controls the wavelength of light output from the laser unit 13 according to operation of the wavelength selecting switch 37.

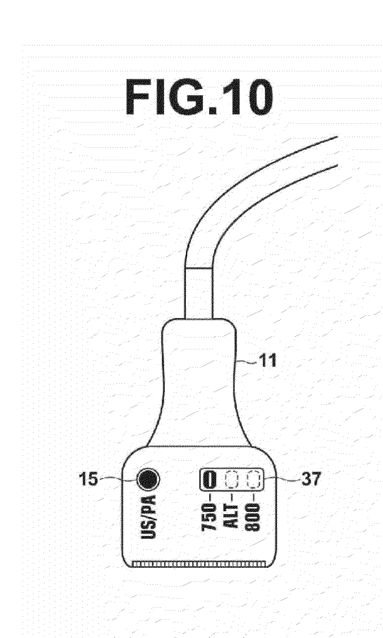

FIG. 10 illustrates the outer appearance of the probe 11. The wavelength selecting switch 37 is provided on the probe 11 in addition to the mode switching switch 15. The wavelength selecting switch 37 is configured as a slide switch, for example. When the slide position of the wavelength selecting switch 37 is at the position “750”, the trigger control circuit 36 causes the laser unit 13 to output light having a wavelength of 750 nm. When the slide position of the wavelength selecting switch 37 is at the position “800”, the trigger control circuit 36 causes the laser unit 13 to output light having a wavelength of 800 nm. When the slide position of the wavelength selecting switch 37 is at the position “ALT”, the trigger control circuit 36 causes the laser unit 13 to alternately output light having a wavelength of 750 nm and light having a wavelength of 800 nm.

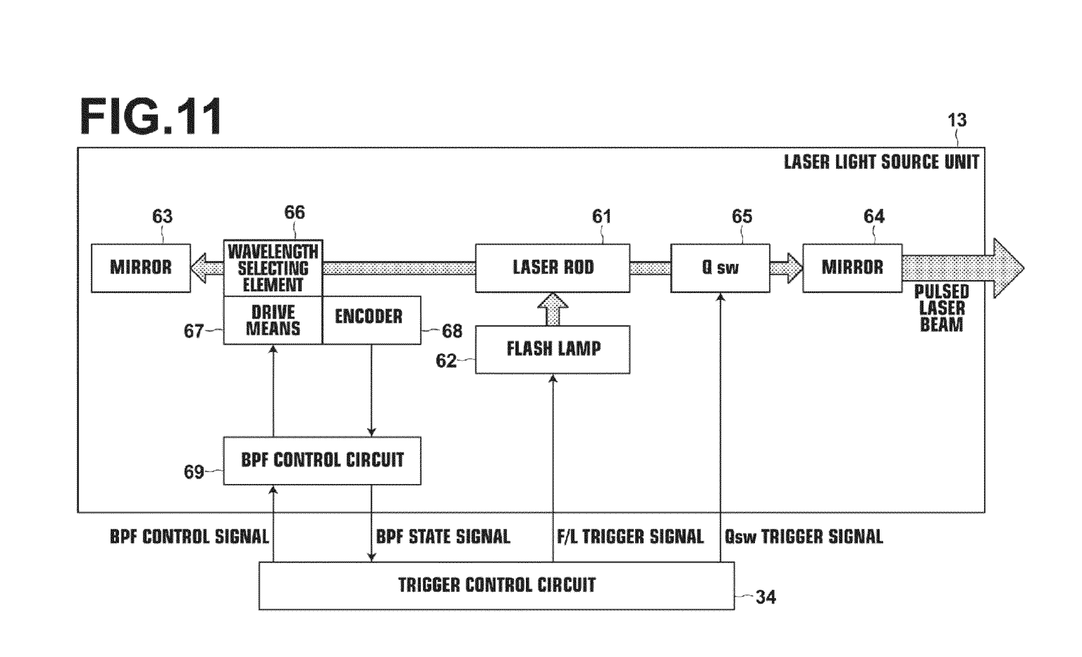

Next, the configuration of the laser unit 13 will be described in detail. FIG. 11 illustrates the construction of the laser unit 13. The laser unit 13 has: a laser rod 61, a flash lamp 62, mirrors 63 and 64, a Q switch 65, a wavelength selecting element 66, a drive means 67, a driving state detecting means 68, and a BPF control circuit 69.

The laser rod 61 is a maser medium. An alexandrite crystal, a Cr:LiSAF (Cr:LiSrAlF6), Cr:LiSAF (Cr:LiCaAlF6) crystal, or a Ti:Sapphire crystal may be employed as the laser rod 61. The flash lamp 62 is a pumping light source, and irradiates pumping light onto the laser rod 61. Light sources other than the flash lamp 62, such as semiconductor lasers and solid state lasers, may be employed as the pumping light source.

The mirrors 63 and 64 face each other with the laser rod 61 sandwiched therebetween. The mirrors 63 and 64 constitute an optical resonator. Here, the mirror 64 is an output side mirror. The Q switch 65 is inserted within the resonator. The Q switch 65 changes the insertion loss within the optical resonator from high loss (low Q) to low loss (high Q) at high speed, to obtain a pulsed laser beam.

The wavelength selecting element 66 includes a plurality of band pass filters (BPF: Band Pass Filters) that transmit wavelengths different from each other. The wavelength selecting element 66 selectively inserts the plurality of band pass filters into the optical path of the optical resonator. The wavelength selecting element 66 includes a first band pass filter that transmits light having a wavelength of 750 nm (central wavelength) and a second band pass filter that transmits light having a wavelength of 800 nm (central wavelength), for example. The oscillating wavelength of the laser beam oscillator can be set to 750 nm by inserting the first band pass filter into the optical path of the optical oscillator, and the oscillating wavelength of the laser beam oscillator can be set to 800 nm by inserting the second band pass filter into the optical path of the optical oscillator.

The drive means 67 drives the wavelength selecting element 66 such that the band pass filters which are inserted into the optical path of the optical resonator are sequentially switched in a predetermined order. For example, if the wavelength selecting element 66 is constituted by a rotatable filter body that switches the band pass filter to be inserted into the optical path of the optical resonator by rotational displacement, the drive means 67 continuously rotates the rotatable filter body. The driving state detecting means 68 detects the rotational displacement of the wavelength selecting element 66, which is a rotatable filter body, for example. The driving state detecting means 68 outputs BPF state data that indicate rotational displacement positions of the rotatable filter body.

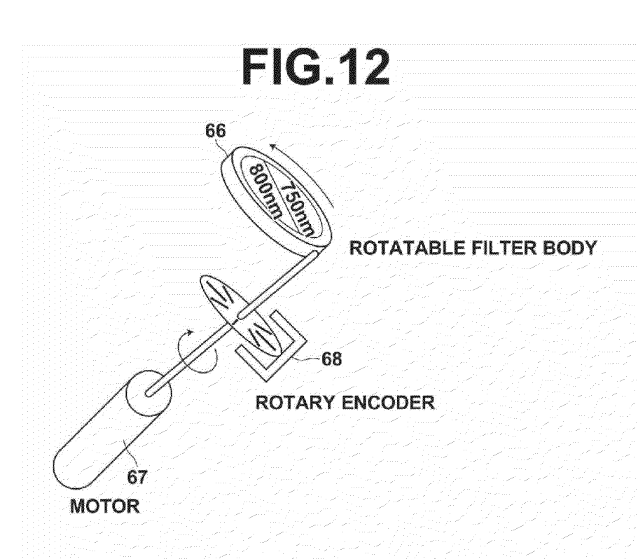

FIG. 12 illustrates an exampled of the configurations of the wavelength selecting element 66, the drive means 67, and the driving state detecting means 68. In this example, the wavelength selecting element 66 is a rotatable filter body that includes two band pass filters, and the drive means is a servo motor. In addition, the driving state detecting means 68 is a rotary encoder. The wavelength selecting element 66 rotates according to rotation of an output shaft of the servo motor. Half of the rotatable filter body (rotational displacement positions from 0° to 180°, for example) is formed as the first band pass filter that transmits light having a wavelength of 750 nm, and the other half of the rotatable filter body (rotational displacement positions from 180° to 360°, for example) is formed as the second band pass filter that transmits light having a wavelength of 800 nm, for example. By rotating such a rotatable filter body, the first band pass filter and the second band pass filter can be alternately inserted into the optical path of the optical resonator at a switching speed corresponding to the rotating speed of the rotatable filter body.

The rotary encoder that constitutes the driving state detecting means 68 detects the rotational displacement of the wavelength selecting element 66, which is a rotatable filter body, with a rotatable plate having a slit mounted on the output shaft of the servo motor, and a transmissive type photo interrupter, and generates BPF state data. The driving state detecting means 68 outputs the BPF state data that represent rotational displacement positions of the rotatable filter body to the BPF control circuit 69.

The BPF control circuit 69 controls voltage which is supplied to the drive means 67 such that the amount of rotational displacement detected by the driving state detecting means 68 within a predetermined amount of time becomes an amount corresponding to a predetermined rotational speed of the rotatable filter body, when alternate output of light having two wavelengths is selected by the wavelength selecting switch 37 (FIG. 9). The trigger control circuit 36 outputs a command that specifies the rotational speed of the rotatable filter body to the BPF control circuit 69, in the form of a BPF control signal. The BPF control circuit 69 monitors the BPF state data and controls the voltage supplied to the servo motor such that the amount of rotational displacement detected by the rotary encoder during a predetermined amount of time is maintained at an amount corresponding to the specified rotational speed, for example. The trigger control circuit 36 may be employed instead of the BPF control circuit 69 to monitor the BPF state data and control the drive means 67 such that the wavelength selecting element 66 is driven at a predetermined speed.

Hereinafter, the operational procedures of an operating mode that generates ultrasound images, an operating mode that generates photoacoustic images, and an operating mode that generates both types of images, will be described. First, the operational procedure of an operating mode that generates ultrasound images will be described. The operational procedures for producing an ultrasound image are basically the same as those described with respect to the first embodiment. The trigger control circuit 36 (FIG. 9) outputs an ultrasonic wave trigger signal to the transmission control circuit 23. The probe 11 transmits ultrasonic waves into the body of a subject. The probe 11 detects reflected acoustic waves which are reflected within the body of the subject. The receiving circuit 16 within the ultrasonic wave unit 12a receives detected signals (reflected acoustic signals) of the reflected acoustic waves. The A/D converting means 17 samples the reflected acoustic signals and stores the sampled reflected acoustic signals in the reception memory 24. The data separating means 25 reads out the reflected acoustic signals from the reception memory 24 and provides the read out reflected acoustic signals to the ultrasound image reconstructing means 32. The reflected acoustic signals are detected and logarithmically converted by the detecting/logarithmic converting means 33 after being reconstructed by the ultrasound image reconstructing means 32. The ultrasound image constructing means 34 generates an ultrasound image based on the detected and logarithmically converted reflected acoustic signals. The generated ultrasound image is displayed on the display screen of the image display means 14.

Next, the operational procedures of the operating mode that generates photoacoustic images will be described. Here, it is assumed that alternate output of light having a wavelength of 750 nm and light having a wavelength of 800 nm is selected by the wavelength selecting switch 37. The trigger control circuit 36 controls the BPF control circuit 69 such that the band pass filters which are inserted into the optical path of the optical resonator within the laser unit 13 by the wavelength selecting element 66 (FIG. 11) are switched at a predetermined switching speed. The trigger control circuit 36 outputs BPF control signals that cause the rotatable filter body that constitutes the wavelength selecting element 66 to rotate continuously in a predetermined direction at a predetermined rotational speed, for example. The rotational speed of the rotatable filter body may be determined based on the number of wavelengths (the number of band pass filters) and the number of pulsed laser beams to be output by the laser unit 13 per unit time.

The trigger control circuit 36 outputs a flash lamp trigger signal to the laser unit 13 that causes the flash lamp 62 to irradiate a pumping light beam onto the laser rod 61. The trigger control circuit 36 outputs the flash lamp trigger signals at predetermined temporal intervals based on BPF state signals. For example, the trigger control circuit 36 outputs a flash lamp trigger signal when the BPF state data represents a position which is the driven position of the wavelength selecting element 66 at which the band pass filter corresponding to the wavelength (the first wavelength) of a pulsed laser beam to be output minus an amount of displacement that the wavelength selecting element will undergo during an amount of time necessary to pump the laser rod, to cause the pumping light beam to be irradiated onto the laser rod 61. The trigger control circuit 36 outputs the flash lamp trigger signals at periodically at predetermined temporal intervals, for example.

After outputting the flash lamp trigger signal, the trigger control circuit 36 outputs a Q switch trigger signal to the Q switch 65 of the laser unit 13. The trigger control circuit 36 outputs the Q switch trigger signal at a timing at which the band pass filter that transmits a wavelength corresponding to the wavelength (the first wavelength) of a pulsed laser beam to be output is inserted into the optical path of the optical resonator. For example, in the case that the wavelength selecting element 66 is constituted by a rotatable filter body, the trigger control circuit 36 outputs the Q switch trigger signal when the BPF state data indicates that a band pass filter corresponding to the wavelength of the pulsed laser beam to be output is inserted into the optical path of the optical resonator. The Q switch 65 changes the insertion loss within the optical resonator from high loss to low loss at high speed in response to the Q switch trigger signal, to output a pulsed laser beam from the output side mirror 64.

The light beam having the first wavelength output by the laser unit 13 is guided to the probe 11, for example, then irradiated onto the subject. The probe 11 detects photoacoustic signals which are generated within living tissue due to irradiation of the laser beam. The receiving circuit 16 within the ultrasonic wave unit 12a receives the photoacoustic signals. The A/D converting means 17 samples the photoacoustic signals which are generated when the first wavelength is irradiated, then stores the sampled photoacoustic signals in the reception memory 24.

Following irradiation of the light having the first wavelength, the trigger control circuit 36 irradiates light having the second wavelength onto the subject by procedures similar to those described above. That is, after a flash lamp trigger signal is output, a Q switch trigger signal is output at a timing at which the band pass filter that transmits a wavelength corresponding to the second wavelength is inserted into the optical path of the optical resonator, to cause the laser unit 13 to output a pulsed laser beam having the second wavelength. The probe 11 detects photoacoustic signals which are generated within living tissue due to irradiation of the laser beam having the second wavelength. The receiving circuit 16 within the ultrasonic wave unit 12a receives the photoacoustic signals. The A/D converting means 17 samples the photoacoustic signals which are generated when the second wavelength is irradiated, then stores the sampled photoacoustic signals in the reception memory 24.

The data separating means 25 reads out the photoacoustic signals which were generated when the light having the first wavelength was irradiated and the photoacoustic signals which were generated when the light having the second wavelength was irradiated from the reception memory, and provides the read out photoacoustic signals to the two wavelength data complexifying means 26. The two wavelength data complexifying means 26 generates complex number in which one of the photoacoustic signals corresponding to the two wavelengths is a real part, and the other is an imaginary part. The photoacoustic image reconstructing means 27 reconstructs the complex number by the Fourier transform method. The two wavelength data calculating means extracts phase data from the reconstructed complex number data. In addition, the intensity data extracting means 29 extracts intensity data from the reconstructed complex number data. The extracted intensity data are detected and logarithmically converted by the detecting/logarithmic converting means 30. The photoacoustic image constructing means 31 generates a photoacoustic image based on the detected and logarithmically converted intensity data and the phase data. The generated photoacoustic image is displayed on the display screen of the image display means.

Next, the operational procedures of the operating mode that generates both ultrasound images and photoacoustic images will be described. Here as well, it is assumed that alternate output of light having a wavelength of 750 nm and light having a wavelength of 800 nm is selected by the wavelength selecting switch 37. The trigger control circuit 36 controls the BPF control circuit 69 such that the band pass filters which are inserted into the optical path of the optical resonator within the laser unit 13 by the wavelength selecting element 66 are switched at a predetermined switching speed in the same manner as in the case described above, in which the photoacoustic image is generated.

The trigger control circuit 36 outputs a flash lamp trigger signal to the laser unit 13, and causes the flash lamp 62 to irradiate a pumping light beam onto the laser rod 61. The trigger control circuit outputs a Q switch trigger signal to the Q switch 65 of the laser unit 13 after outputting the flash lamp trigger signal. The Q switch 65 changes the insertion loss within the optical resonator from high loss to low loss at high speed in response to the Q switch trigger signal, to output a pulsed laser beam from the output side mirror 64.

The light beam having the first wavelength output by the laser unit 13 is guided to the probe 11, for example, then irradiated onto the subject. The probe 11 detects photoacoustic signals which are generated within living tissue due to irradiation of the laser beam. The receiving circuit 16 within the ultrasonic wave unit 12a receives the photoacoustic signals. The A/D converting means 17 samples the photoacoustic signals which are generated when the first wavelength is irradiated, then stores the sampled photoacoustic signals in the reception memory 24.

Following irradiation of the light having the first wavelength, the trigger control circuit 36 irradiates light having the second wavelength onto the subject by procedures similar to those described above. That is, after a flash lamp trigger signal is output, a Q switch trigger signal is output at a timing at which the band pass filter that transmits a wavelength corresponding to the second wavelength is inserted into the optical path of the optical resonator, to cause the laser unit 13 to output a pulsed laser beam having the second wavelength. The probe 11 detects photoacoustic signals which are generated within living tissue due to irradiation of the laser beam having the second wavelength. The receiving circuit 16 within the ultrasonic wave unit 12a receives the photoacoustic signals. The A/D converting means 17 samples the photoacoustic signals which are generated when the second wavelength is irradiated, then stores the sampled photoacoustic signals in the reception memory 24.

When sampling of the photoacoustic signals is complete, the trigger control circuit 36 outputs an ultrasonic wave trigger signal to the transmission control circuit 23. The probe 11 transmits ultrasonic waves into the body of a subject. The probe 11 detects reflected acoustic waves which are reflected within the body of the subject. The receiving circuit 16 within the ultrasonic wave unit 12a receives detected signals (reflected acoustic signals) of the reflected acoustic waves. The A/D converting means 17 samples the reflected acoustic signals and stores the sampled reflected acoustic signals in the reception memory 24.

The data separating means 25 reads out the photoacoustic signals which were generated when the light having the first wavelength was irradiated and the photoacoustic signals which were generated when the light having the second wavelength was irradiated from the reception memory, and provides the read out photoacoustic signals to the two wavelength data complexifying means 26. In addition, the data separating means 25 reads out the reflected acoustic signals from the reception memory 24, and provides the read out reflected acoustic signals to the ultrasound image reconstructing means 32.

The two wavelength data complexifying means 26 generates complex number in which one of the photoacoustic signals corresponding to the two wavelengths is a real part, and the other is an imaginary part. The photoacoustic image reconstructing means 27 reconstructs the complex number by the Fourier transform method. The two wavelength data calculating means extracts phase data from the reconstructed complex number data. In addition, the intensity data extracting means 29 extracts intensity data from the reconstructed complex number data. The extracted intensity data are detected and logarithmically converted by the detecting/logarithmic converting means 30. The photoacoustic image constructing means 31 generates a photoacoustic image based on the detected and logarithmically converted intensity data and the phase data.

The reflected acoustic signals provided to the ultrasound image reconstructing means 32 from the data separating means 25 are detected and logarithmically converted by the detecting/logarithmic converting means 33 after being reconstructed by the ultrasound image reconstructing means 32. The ultrasound image constructing means 34 generates an ultrasound image based on the detected and logarithmically converted reflected acoustic signals. The photoacoustic image generated by the photoacoustic image constructing means 31 and the ultrasound image generated by the ultrasound image constructing means 34 are combined by the image combining means 35, then displayed on the display screen of the image display means 14.

In the present embodiment, the laser unit 13 includes the wavelength selecting element 6, and the laser unit 13 is capable of irradiating a plurality of laser beams having different wavelengths from each other onto subjects. When the wavelength selecting element 66 includes two band pass filters that transmit different wavelengths, the wavelength of light output by the laser unit can be controlled by selectively inserting the band pass filters into the optical path of the optical resonator. In addition, laser beams having different wavelengths can be continuously switched and output by the laser unit 13b, by continuously driving the wavelength selecting element that includes two band pass filters that transmit different wavelengths, to continuously and selectively insert the two band pass filters into the optical path of the optical resonator, for example. Functional imaging that utilizes the fact that light absorption properties of light absorbers differ according to wavelengths is enabled by employing photoacoustic signals (photoacoustic data) obtained by irradiating pulsed laser beams having different wavelengths.

In the present embodiment, complex number data, in which one of the first photoacoustic data and the second photoacoustic data is designated as a real part and the other is designated as an imaginary part, are generated, and a reconstructed image is generated from the complex number data by the Fourier transform method. In such a case, only a single reconstruction operation is necessary, and reconstruction can be performed more efficiently compared to a case in which the first photoacoustic data and the second photoacoustic data are reconstructed separately.

In the present embodiment, the photoacoustic measuring apparatus 10a includes the wavelength selecting switch 37 for selecting the wavelength of light to be irradiated onto subjects. Users are enabled to select the wavelength of light beams which are output from the laser unit 13 by operating the wavelength selecting switch 37. In the present embodiment, the wavelength selecting switch 37 is provided on the probe 11, and users can switch the wavelength of light to be irradiated onto subjects without removing the probe 11 from their hands. The advantageous effect that switching of operating modes among operating modes that generate ultrasound images and operating modes that generate photoacoustic images is facilitated by operating the mode switching switch 15 provided on the probe 11 is the same as that of the first embodiment.

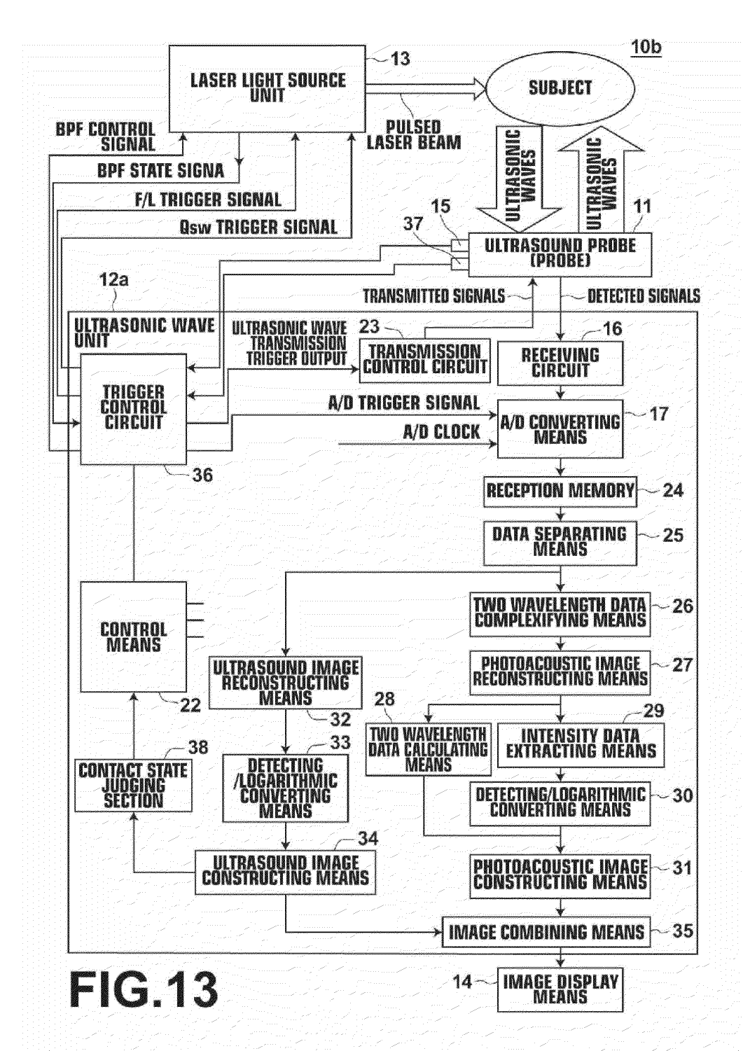

Next, a third embodiment of the present invention will be described. FIG. 13 illustrates a photoacoustic measuring apparatus 10b according to the third embodiment of the present invention. The photoacoustic measuring apparatus 10b of the present embodiment has a contact state judging means 38 within an ultrasonic wave unit 12b in addition to the structures of the photoacoustic measuring apparatus 10a of the second embodiment. The contact state judging means 36 judges whether the probe 11 is in contact with a subject. The trigger control circuit 36 outputs a flash lamp trigger signal to the laser unit 13 when the contact state judging means 38 judges that the probe 11 is in contact with a subject, to irradiate light onto the subject.

In the present embodiment, the probe transmits acoustic waves prior to light being irradiated onto subjects. The probe 11 detects reflected acoustic signals of transmitted ultrasonic waves. The contact state judging means 38 judges whether the probe 11 is in contact with a subject based on the reflected acoustic signals detected by the probe 11. More specifically, the contact state judging means 38 employs ultrasound images generated by the ultrasound image constructing means 34 based on the reflected acoustic signals to judge whether the probe 11 is in contact with a subject.

The contact state judging means 38 has stored therein a typical ultrasound image generated in a state in which the probe 11 is not in contact with the subject as a reference image, for example. The contact state judging means 38 compares the ultrasound image generated by the ultrasound image constructing means 34 against the stored reference image, and judges whether the probe 11 is in contact with the subject based on the results of the comparison. The contact state judging means 38 calculates a degree of similarity between the ultrasound image generated by the ultrasound image constructing means 34 and the reference image, for example. The contact state judging means 38 judges that the probe 11 is not in contact with the subject when the degree of similarity between the two ultrasound images is a predetermined threshold value or greater. The contact state judging means 38 judges that the prove 11 is in contact with the subject if the degree of similarity is less than the threshold value. The trigger control circuit suppresses output of at least one of the flash lamp trigger signal and the Q switch trigger signal when the contact state judging means 38 judges that the probe is not in contact with the subject, thereby preventing a laser beam from being irradiated onto the subject from the probe 11.

A case was described above in which the contact state judging means 38 has the reference image stored therein, and judges whether the probe 11 is in contact with the subject based on the degree of similarity with the reference image. However, the method by which the contact state is judged based on the ultrasound image is not limited to this case. For example, the contact state judging means 38 may perform feature analysis of the ultrasound image generated by the ultrasound image constructing means 34, and may judge whether the probe 11 is in contact with the subject based on the results of the feature analysis. Commonly, saturated high brightness lines are arrayed parallel to the ultrasonic transducers in an ultrasound image which is generated in a state in which the probe 11 is not in contact with a subject. The contact state judging means 38 may judge that a generated ultrasound image was generated in a state in which the probe 11 is not in contact with a subject in the case that saturated high brightness lines are arrayed parallel to the ultrasonic transducers in the generated ultrasound image.

The contact state judging means 38 may judge the contact state based on the signal waveform of the reflected acoustic signals instead of judging the contact state based on ultrasound images. For example, the contact state judging means 38 performs feature analysis of the signal waveform of the reflected acoustic signals, and judges whether the characteristics of a signal waveform of reflected acoustic signals which are observed when the probe 11 is not in contact with a subject are present in the signal waveform of the reflected acoustic signals sampled by the A/D converting means 17 (reflected acoustic signals read out from the reception memory 24). For example, the contact state judging means 38 checks how many locations at which the amplitude of the signal level is greater than or equal to a predetermined level corresponding to a saturation level, and also checks the intervals among locations at which the signal levels are saturated. The contact state judging means 38 may judge that the probe 11 is not in contact with the subject in cases that a plurality of locations at which the signal levels of the reflected acoustic signals are at saturation level are arranged at equidistant intervals, for example. Conversely, the contact state judging means 38 may judge that the probe 11 is in contact with the subject in the case that locations at which signal levels are at saturation level are not arranged at equidistant intervals.

The judgment of contact states based on the signal waveform of reflected acoustic signals is not limited to that described above. For example, the contact state judging means 38 may have a typical signal waveform of reflected acoustic signals when the probe 11 is not in contact with a subject stored therein as a reference waveform. The contact state judging means 38 may calculate correlations between the reference signal waveform and the signal waveform of the reflected acoustic signals output by the A/D converting means 17, and judge how similar the images are based on the calculated correlations. In this case, the contact state judging means 38 administers a threshold value process on the degree of similarity between the two signal waveforms, judges that the probe 11 is not in contact with the subject if the degree of similarity is high, and judges that the probe 11 is in contact with the subject if the degree of similarity is low.

Note that signals output by the ultrasound image reconstructing means 32, or the detecting/logarithmic converting means 33 may be input to the contact state judging means 38 instead of the reflected acoustic signals sampled by the A/D converting means 17. In these cases as well, it is possible to judge whether the probe 11 is in contact with a subject based on the signal waveform of the reflected acoustic signals.

Note that the second and third embodiments were described as examples in which the first photoacoustic data and the second photoacoustic data were complexified. Alternatively, the first photoacoustic and the second photoacoustic data may be reconstructed separately without administering the complexifying operation. In addition, the reconstruction method is not limited to the Fourier transform method. Further, the second and third embodiments calculate the ratio between the first photoacoustic data and the second photoacoustic data by employing the phase data obtained by the complexifying operation. However, the same effects can be obtained by calculating the ratio using the intensity data of the first and second photoacoustic data. In addition, the intensity data may be generated based on signal intensities within a first reconstructed image and signal intensities within a second reconstructed image.

The number of pulsed laser beams having different wavelengths which are irradiated onto a subject when generating photoacoustic images is not limited to two. Three or more pulsed laser beams may be irradiated onto the subject, and photoacoustic images may be generated based on photoacoustic data corresponding to each wavelength. In this case, the two wavelength data calculating means 28 may generate the relationships among signal intensities of photoacoustic data corresponding to each wavelength as phase data. In addition, the intensity data extracting means 29 may generate a sum of signal intensities of photoacoustic data corresponding to each wavelength as intensity data.

The second embodiment was described mainly as a case in which the wavelength selecting element 66 is constituted by a rotatable filter body having two band pass filter regions. However, it is only necessary for the wavelength selecting element 66 to be that which can change the wavelength of light that oscillates within the optical resonator, and is not limited to a rotatable filter body. For example, the wavelength selecting element may be constituted by a rotatable body having a plurality of band pass filters provided on the circumference thereof. It is not necessary for the wavelength selecting element 66 to be a rotatable body. For example, a plurality of band pass filters may be arranged in a row. In this case, the wavelength selecting element 66 may be driven such that the plurality of band pass filters are cyclically inserted into the optical path of the optical resonator, or the wavelength selecting element 66 may be reciprocally driven such that the plurality of band pass filters arranged in a row traverse the optical path of the optical resonator. As a further alternative, a wavelength selecting element such as a birefringent filter may be employed instead of the band pass filters. In addition, when selecting between two wavelengths, if the gains of the wavelengths are different, long pass filters or short pass filters may be utilized instead of the bandpass filters. For example, in the case that an alexandrite laser outputs laser beams having wavelengths of 800 nm and 750 nm, selection of each wavelength is possible by utilizing a combination of long pass filters for 800 nm and 750 nm, because the gain of the 750 nm laser beam is greater.

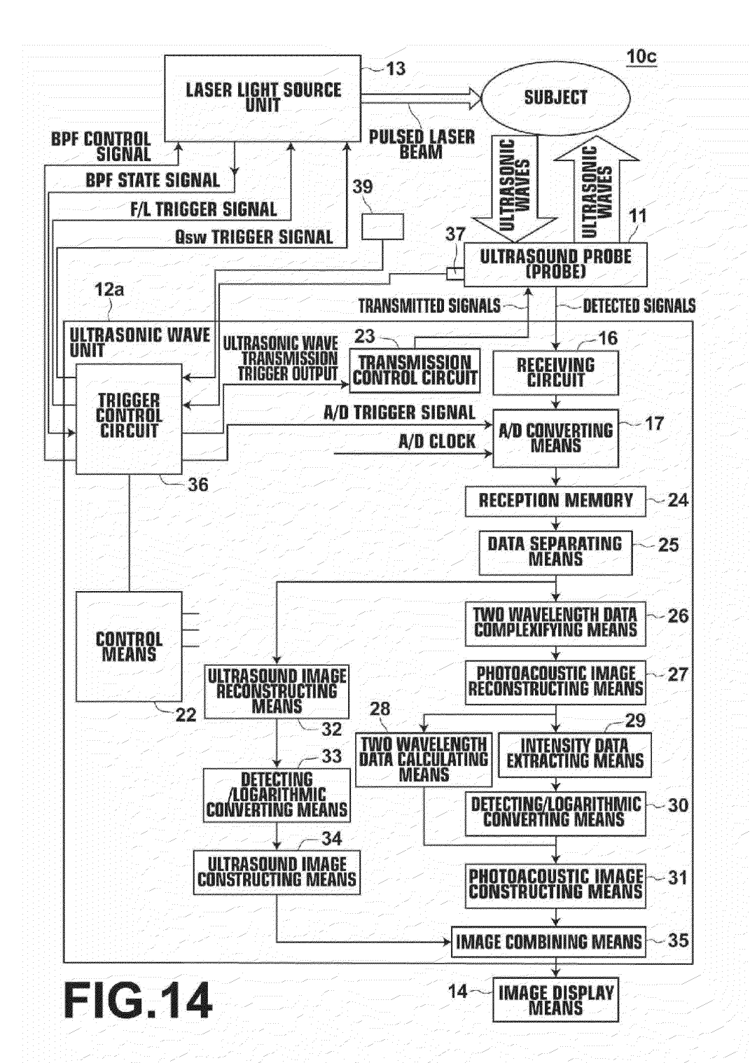

The second and third embodiments were described as cases in which the mode switching switch 15 and the wavelength selecting switch 37 were both provided on the probe 11. However, only at least one of the mode switching switch 15 and the wavelength selecting switch 37 need to be provided on the probe 11, and it is not necessary for both switches to be provided on the probe 11. FIG. 14 illustrates a photoacoustic measuring apparatus 10c according to a modification of the present invention. The mode switching switch 15 is provided on the probe 11 in the photoacoustic measuring apparatus 10a of the second embodiment. In contrast, a footswitch 39 is employed as a switch for switching operating modes of the apparatus in FIG. 14. The footswitch (mode switching switch) 39 operates alternately, for example, and the operating mode of the apparatus is switched among operating modes that include detection of photoacoustic signals and operating modes that do not include detection of photoacoustic signals each time that the footswitch 39 is operated.

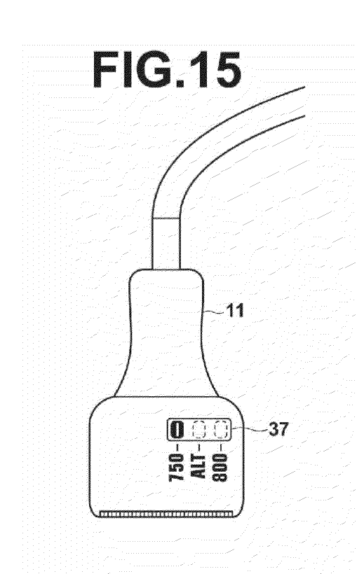

FIG. 15 illustrates the outer appearance of the probe 11 of the modification of the present invention. Only the wavelength selecting switch 37 constituted by a slide switch is provided on the probe 11, and the mode switching switch 15 which is present in the probe 11 of the second embodiment illustrated in FIG. 10 is not provided. In the case that such a configuration is adopted, users may select the wavelength of light to be irradiated onto subjects by operating the wavelength selecting switch 37 with their hands. Meanwhile, generation of ultrasound images and generation of photoacoustic images can be switched by operating the footswitch 39 with their feet.

Preferred embodiments of the present invention have been described above. However, the photoacoustic measuring apparatus and the photoacoustic image generating method are not limited to the above embodiments. Various changes and modifications to the configurations of the above embodiments are included in the scope of the present invention.

What is claimed is:

1. A photoacoustic measuring apparatus, comprising:
a light source;
an acoustic wave transmitting section that transmits acoustic waves toward a subject;
a light irradiating section that irradiates a light beam guided from the light source toward the subject;
a probe including an acoustic wave detecting section that detects photoacoustic waves generated within the subject due to irradiation of the light beam onto the subject and detects reflected acoustic waves of the acoustic waves transmitted into the subject;
a mode switching switch provided on the probe; and
a control section that switches among operating modes in which the acoustic wave detecting section detects at least the photoacoustic waves, and operating modes in which the acoustic wave detecting section does not detect the photoacoustic waves, in response to operation of the mode switching switch.

2. A photoacoustic measuring apparatus as defined in claim 1, wherein:
the operating modes include a first operating mode in which the acoustic wave detecting section detects the photoacoustic waves, a second operating mode in which the acoustic wave detecting section detects the reflected acoustic waves, and a third operating mode in which the acoustic wave detecting section detects both the photoacoustic waves and the reflected acoustic waves; and
the control section switches among the first through third operating modes each time that the mode switching switch is operated.

3. A photoacoustic measuring apparatus as defined in claim 1, wherein:
the control section sets the operating mode to an operating mode in which the acoustic wave detecting section does not detect the photoacoustic waves in an initial state.

4. A photoacoustic measuring apparatus as defined in claim 1, wherein:
the mode switching switch is an alternate action push button switch.

5. A photoacoustic measuring apparatus as defined in claim 1, further comprising:
an image generating section that generates photoacoustic images and acoustic images based on detected signals of the photoacoustic waves and detected signals of the reflected acoustic waves.

6. A photoacoustic measuring apparatus as defined in claim 1, wherein:
the probe includes at least one of the acoustic wave transmitting section and the light irradiating section.

7. A photoacoustic measuring apparatus as defined in claim 1, wherein:
the light source is capable of outputting light beams having a plurality of different wavelengths;
the photoacoustic measuring apparatus further comprises a wavelength selecting switch for selecting the wavelength of a light beam to be irradiated onto a subject; and
the control section controls the wavelength of the light beam to be output from the light source in response to operation of the wavelength selecting switch, in addition to switching of the operating modes.

8. A photoacoustic measuring apparatus as defined in claim 7, wherein:
the wavelength selecting switch is provided on the probe.

9. A photoacoustic measuring apparatus as defined in claim 7, wherein:
the control section causes the light source to output alight beam having a first wavelength, to output a light beam having a second wavelength different from the first wavelength, or to alternately output the light beam having the first wavelength and the light beam having the second wavelength, in response to operation of the wavelength selecting switch.

10. A photoacoustic measuring apparatus as defined in claim 7, wherein:
the wavelength selecting switch is a slide switch.

11. A photoacoustic measuring apparatus as defined in claim 1, further comprising:
a contact state judging section that judges whether the probe is in contact with a subject; and wherein:
a light beam is irradiated onto the subject when the contact state judging section judges that the probe is in contact with the subject.

12. A photoacoustic measuring apparatus as defined in claim 11, wherein:
the contact state judging section judges whether the probe is in contact with the subject, based on detected signals of the reflected acoustic waves.

13. A photoacoustic measuring apparatus as defined in claim 1, wherein:
the control section sets the operating mode to an operating mode that in which the acoustic wave detecting section detects only the reflected acoustic waves from among the photoacoustic waves and the reflected acoustic waves in an initial state.

14. A photoacoustic measuring apparatus as defined in claim 1, wherein:
the control section further outputs a signal to the light source that controls the output of light from the light source.

15. A photoacoustic measuring apparatus, comprising:
a light source;
an acoustic wave transmitting section that transmits acoustic waves toward a subject;
a light irradiating section that irradiates a light beam guided from the light source toward the subject;
a probe including an acoustic wave detecting section that detects photoacoustic waves generated within the subject due to irradiation of the light beam onto the subject and detects reflected acoustic waves of the acoustic waves transmitted into the subject;
a mode switching switch for switching operating modes;
a wavelength selecting switch for selecting the wavelength of the light beam to be output from the light source; and
a control section that switches among operating modes in which the acoustic wave detecting section detects at least the photoacoustic waves, and operating modes in which the acoustic wave detecting section does not detect the photoacoustic waves in response to operation of the mode switching switch, and controls the wavelength of the light beam which is output from the light source in response to operation of the wavelength selecting switch;
at least one of the mode switching switch and the wavelength selecting switch being provided on the probe.

16. A photoacoustic measuring apparatus as defined in claim 15, wherein:
both the mode switching switch and the wavelength selecting switch are provided on the probe.

17. A photoacoustic measuring apparatus as defined in claim 15, wherein:
the mode switching switch is an alternate action push button switch.

18. A photoacoustic measuring apparatus as defined in claim 15, wherein:
only the wavelength selecting switch is provided on the probe; and
the mode switching switch is a footswitch to be operated by the foot of an operator.

19. A photoacoustic measuring apparatus as defined in claim 18, wherein:
the footswitch is an alternate action switch.

20. A photoacoustic measuring apparatus as defined in claim 15, wherein:
the wavelength selecting switch is a slide switch.
